(19) United States
(12) Patent Application Publication
(10) Pub. No.: US 2023/0165637 A1
(43) Pub. Date: Jun. 1, 2023

(54) BIPOLAR CATHETER FOR VASCULAR TREATMENT

(71) Applicant: Biounit Co., LTD., Hanam-si (KR)

(72) Inventor: KYUNG TAE KIM, Seoul (KR)

(21) Appl. No.: 18/160,585

(22) Filed: Jan. 27, 2023

Related U.S. Application Data
(63) Continuation of application No. PCT/KR2021/009775, filed on Jul. 28, 2021.

(30) Foreign Application Priority Data
Jul. 29, 2020 (KR) 10-2020-0094438

(51) Int. Cl. A61B 18/14

(52) U.S. Cl. CPC A61B 18/1492; A61B 2018/00595

(57) ABSTRACT

The present disclosure relates to a bipolar catheter which enables a more precise surgical procedure by being configured to be able to evenly apply a high-frequency wave by an electrode tip being rotated inside a lesion area, and which can minimize damage to a surrounding tissue that can occur during an invasive surgery using a catheter, and which also enables a safer and more accurate surgical procedure compared to an existing surgical procedural method dependent on the medical technique of an operator, by electronically precisely controlling the position of the electrode tip within the lesion area.

### CROSS-REFERENCE TO RELATED APPLICATIONS

This application is a Continuation of Application No. PCT/KR2021/009775, filed on Jul. 28, 2021, which in turn claims the benefit of Korean Patent Application No. 10-2020-0094438, filed on Jul. 29, 2020. The entire disclosures of all these applications are hereby incorporated by reference.

### TECHNICAL FIELD

The present disclosure relates to a bipolar catheter for a vascular treatment, and more particular, to a bipolar catheter for a vascular treatment which may effectively come into contact with a lesion area by rotating an electrode tip inside the lesion area, which enables a pull-back function of the electrode tip through electronic control to prevent a portion to which high frequency is applied from being overlapping by a backward movement of the electrode tip or prevent the occurrence of a non-contact portion to which the high frequency is not transmitted, and which may minimize the number of electrodes and reduce a diameter of an electrode unit, thereby enabling more precise procedures and minimizing damage to surrounding tissue that may occur during invasive surgery with a catheter.

### BACKGROUND ART

In general, when a lesion area occurs in a body organ, it is treated with a surgical operation or procedure. The surgical procedure may be understood as a non-surgical concept, and since such non-surgical procedures have a lower risk than surgical procedures and have a low incidence of surgical trauma procedure, and may be performed relatively simply, non-surgical methods are frequently used except for indications that necessarily require surgical surgery.

Specifically, a representative disease among non-surgical vascular diseases is, for example, varicose vein disease.

A varicose vein is a disease caused by the reflux of blood that has to flow to the heart to the lower extremity due to abnormal function of a valve in leg veins, and refers to a disease in which the skin of legs twists and swells while the legs swell, and venous blood vessels dilate or stretch. Such varicose veins may result in complications such as pigmentation, nerve damage, wound infection, skin scarring, capillary dilatation matting, recurrence of reflux, deep vein, thrombosis, and pulmonary embolism.

Treatments for the varicose veins may include a traditional surgical method of ligation of the vein through skin incision, an injection sclerosis treatment of injecting intravascular sclerosis, or a recent minimally invasive method such as intravenous closure using laser and high frequency, or the like, and a method of blocking and closing veins using biological adhesives is also being used.

However, among these treatments, the minimally invasive method using laser or high frequency is recognized as a treatment method in which vascular treatment is the first priority, in consideration of increased convenience of treatment, shorter recovery period, and cosmetic effects due to minimization of surgical scars, and the like.

Among them, an invasive method using high frequency electrodes is the most effective, and accordingly, doctors and patients require the aforementioned method.

A representative conventional technology related to the conventional high-frequency invasive treatment device is introduced as follows.

Korean Unexamined Patent Application Publication No. 10-2013-0128926 discloses a high-frequency thermal treatment device (hereinafter referred to as ‘a prior art’). The prior art includes a high frequency generator generating a high frequency current and an electrode for cauterizing a lesion area with a high frequency current generated by the high frequency generator.

The prior art constructed in this way treats the lesion area by inserting an electrode into a body and radiating high frequency energy therein.

However, the prior art may not control a cauterization length of the electrode, and may make it inevitable to generate an alternating interval between an active electrode body and a passive electrode body. Accordingly, there is a limit to uniformly dissipating heat through high frequency due to a pitch void, which may make it impossible to uniformly radiate heat energy to the lesion area.

On the other hand, in consideration of such a problem, a plurality of high frequency thermal treatment devices with different lengths of electrodes, and depending on the lesion area, the high frequency thermal treatment device having an electrode of which the length is suitable for the lesion area may be used. However, in this case, there may be a problem in that the costs consumed to provide the plurality of high frequency thermal treatment devices may increase, and the high frequency thermal treatment device has to be replaced according to the size of the lesion area.

In addition, since there was a limit to making the diameter of the electrode thin, it was difficult for an electrode tip to get into vein blood vessels which was severely curvy, and in the operation of the electrode, even by reversing the electrode with the experience of the clinician and ultrasound imaging, as illustrated in FIG. 11, portions to which the high frequency is applied overlapped each other (an overlapping portion) or a portion where the high frequency cannot be transmitted (a non-contact portion) occurred, which had difficulty conducting uniform heat treatment.

In addition, since an operator has to manually reverse the electrode and close target blood vessels, manual operation may not maintain a uniform speed, and when closed vessels have a long length, the treatment time may increase and the operator's fatigue may increase along.

### Prior Art Document

(Patent Document 1) Korean Unexamined Patent Application Publication No. 10-2013-0128926

### DISCLOSURE

### Technical Problem

The present disclosure has been derived to solve the aforementioned problems of the prior art, and objects of the present disclosure provide a bipolar catheter for a vascular treatment which may effectively come into contact with a lesion area by rotating an electrode tip inside the lesion area, which enables a pull-back function of the electrode tip through electronic control to prevent a portion to which high frequency is applied from being overlapping by a backward movement of the electrode tip or prevent the occurrence of a non-contact portion to which the high frequency is not transmitted, and which may minimize the number of electrodes and reduce a diameter of an electrode unit, thereby enabling more precise procedures and minimizing damage to surrounding tissue that may occur during invasive surgery with a catheter.

### Technical Solution

According to one aspect of the present disclosure to achieve the objects, a bipolar catheter for a vascular treatment may include: a base portion; a guide portion installed slidably in the base portion and equipped with a driving motor configured to generate rotational driving force; an electrode unit configured to rotate in conjunction with the driving motor and inserted into a lesion area to cauterize the lesion area; and a driver configured to slide the guide portion in front and rear directions in the base portion.

In addition, according to the preferred embodiment, the guide portion may include: a main body integrated with a lower plate connected to an upper surface of the base portion and equipped with the driving motor, wherein an output end of the driving motor is exposed to the front; and a gripping clip spaced apart from the front of the main body and configured to grip an outer circumferential surface of the electrode unit.

In addition, according to the preferred embodiment, the electrode unit may include: an electrode body to which a high frequency current is applied and in which an electrode tip heated by the high frequency current is formed in one end thereof; an insulating cover formed of a hollow body accommodating the electrode body inside and configured to expose the electrode tip; and a fixing holder formed of a hollow body accommodating the other portion of the insulating cover inside and fitted and inserted into the gripping clip.

In addition, according to the preferred embodiment, the depth of an insertion of the electrode tip into the lesion area may be controlled depending on a sliding distance of the guide portion in front and rear directions.

In addition, according to the preferred embodiment, an outer diameter of the electrode tip may be formed smaller than an inner diameter of the insulating cover.

### Advantageous Effect

The present disclosure has the following effects.

First, when an electrode body rotates by constraining a fixing holder to a gripping clip and configuring the electrode body to freely and relatively rotate inside the fixing holder by rotational driving force of a driving motor, a lesion can be treated by rotating the electrode tip exactly at a target lesion area by implementing a state in which only the electrode tip exposed to one end of an insulating cover rotates when viewed from the outside. Accordingly, according to the present disclosure, a more precise procedure can be performed, and damage to surrounding tissues that may occur during an invasive procedure using a catheter may be minimized.

Second, by electronically controlling a position of the electrode tip in the lesion area, the present disclosure provides the effect of enabling safe and accurate procedures compared to the conventional procedure method that relied on the operator's medical treatment.

Third, when a roller member is simply rotated to move an insulating cover forward and backward without the need to replace electrode tips of different sizes, the exposure length of the electrode tip can be changed by the insulating cover, thereby enabling precise procedures without the need to replace the electrode tip depending on the lesion area. Accordingly, the convenience of treatment can be greatly improved, and peripheral devices of the catheter can be greatly simplified.

Fourth, since the electrode tip can be rotated, even if the number of electrodes (wires) around the electrode tip is reduced, it is possible to effectively radiate high-frequency radiation to the lesion. Accordingly, the diameter of the electrode tip can be minimized and inserted into a small vessel. In addition, when inserting the electrode tip from a central vessel into a branch vessel, or when inserting the electrode tip into a heavily curved vessel, it is possible to reduce vascular wall damage by reducing vascular wall contact.

### DESCRIPTION OF DRAWINGS

FIG. 1 is a view illustrating an overall appearance of a bipolar catheter according to the present disclosure;

FIG. 2 is a view illustrating a decomposition configuration of an electrode unit;

FIG. 3 is a view illustrating a front surface of the bipolar catheter illustrated in FIG. 1;

FIG. 4 is a view schematically illustrating a cross-sectional configuration based on line A-A illustrated in FIG. 3;

FIG. 5 is a view illustrating a state in which a guide portion slides forward based on FIG. 4;

FIG. 6 partially illustrates a form in which an exposure length of an electrode tip is changed by moving an insulating cover forward by an operation of a roller member with respect to FIG. 4;

FIG. 7 is an enlarged view of part B shown in FIG. 4;

FIG. 8A is a view schematically illustrating a cross-sectional configuration based on line C-C illustrated in FIG. 7, and FIG. 8B is a view illustrating a state in which a cylinder illustrated in FIG. 8A is rotated at a certain angle;

FIG. 9 is a view schematically illustrating a cross-sectional configuration based on line D-D illustrated in FIG. 7;

FIG. 10 is a view for explaining another embodiment of a bipolar catheter according to the present disclosure; and

FIG. 11 is a diagram exemplarily illustrating a situation in which a high-frequency overlapping portion and a non-contact portion of a high-frequency occur in a lesion area.

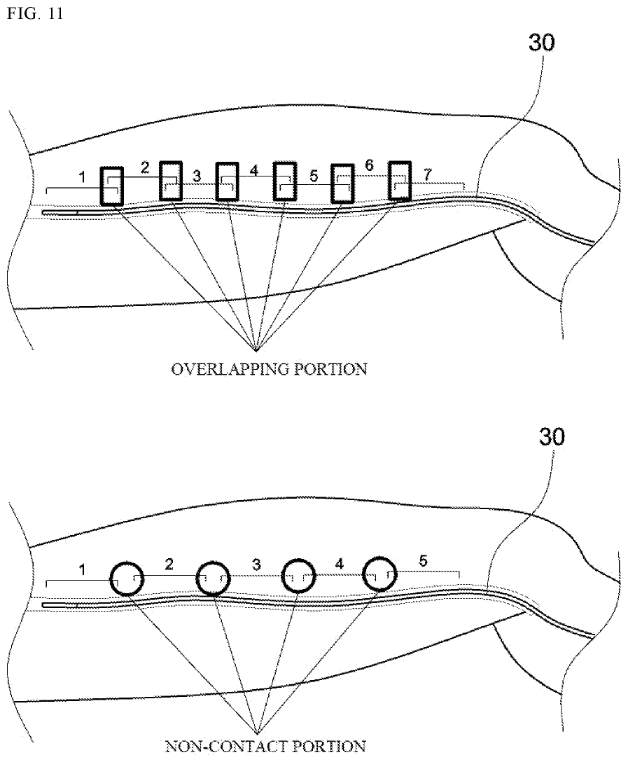

### MODE FOR DISCLOSURE

Hereinafter, embodiments will be described in detail with reference to the annexed drawings for better understanding. However, it will be apparent that the embodiments may be modified in various ways and the scope of the embodiments should not be construed as being limited to the following description. Thus, the embodiments are provided to ensure more perfect comprehension of the embodiments by one of ordinary skill in the art. For clarity, shapes, etc. of respective constituent members illustrated in the drawings may be exaggerated. Wherever possible, the same reference numbers will be used throughout the drawings to refer to the same or like parts. In the following description of the present disclosure, a detailed description of known functions and configurations incorporated herein will be omitted when it may obscure the subject matter of the present disclosure.

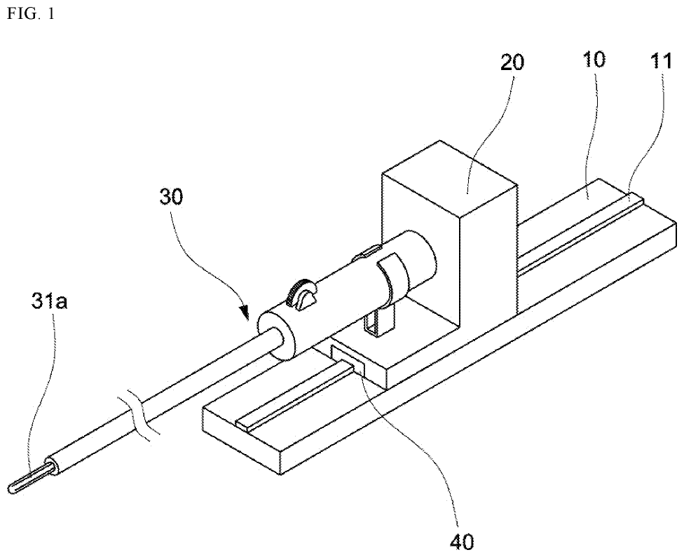

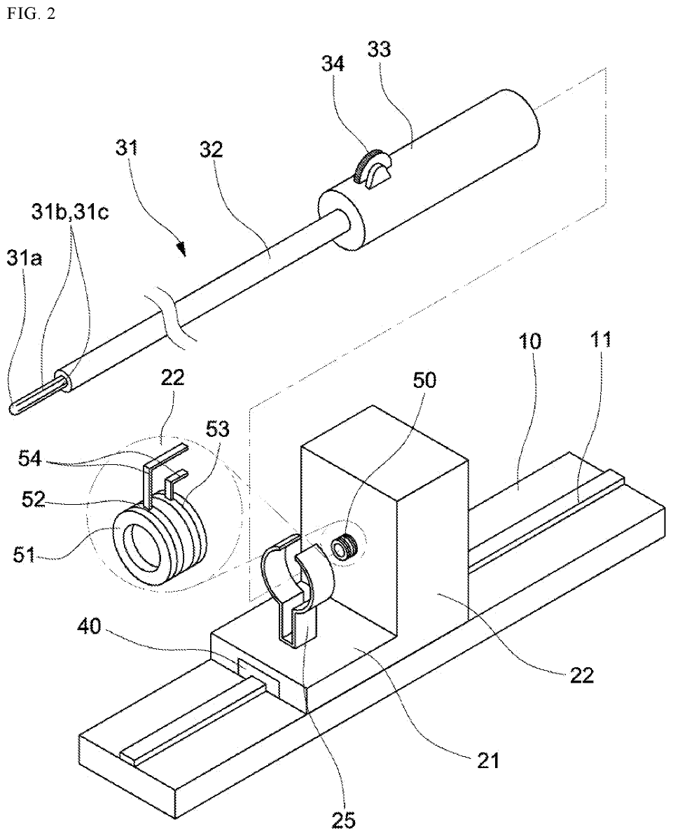

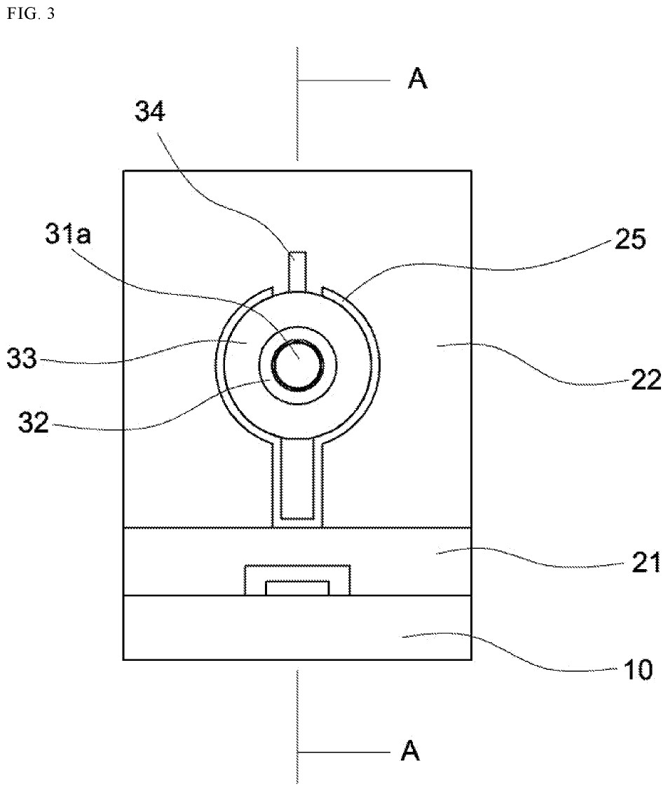

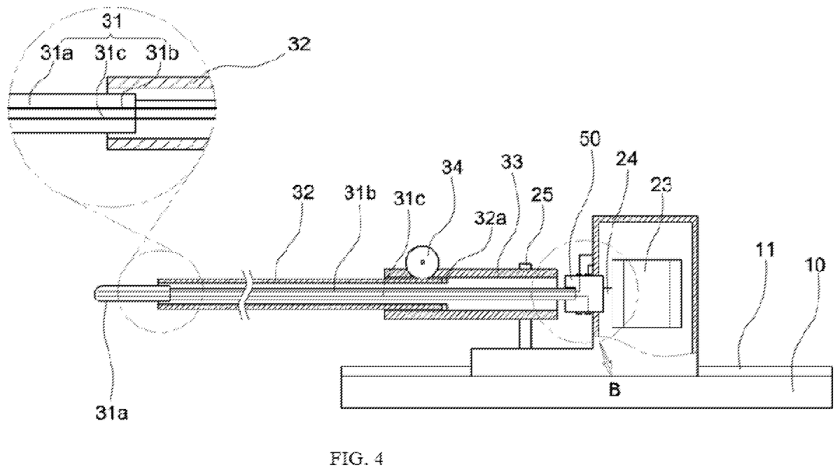

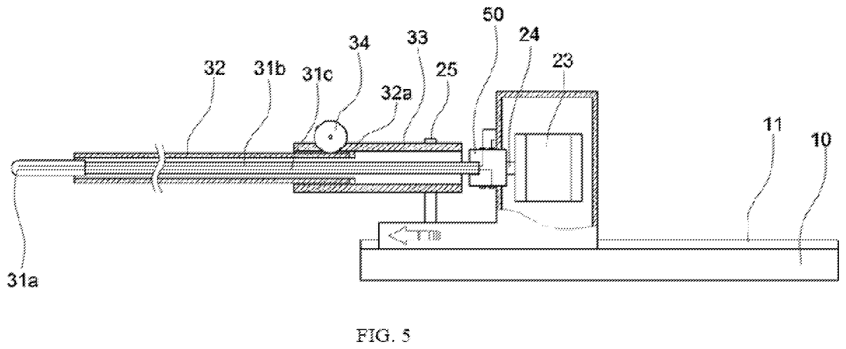

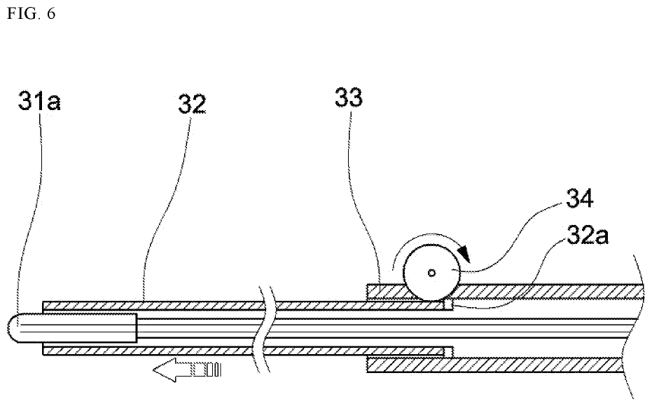

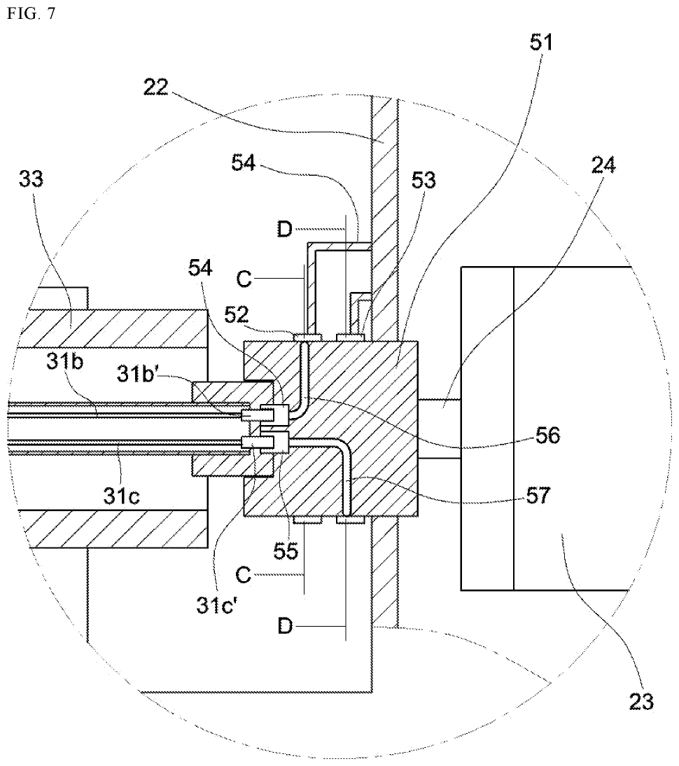

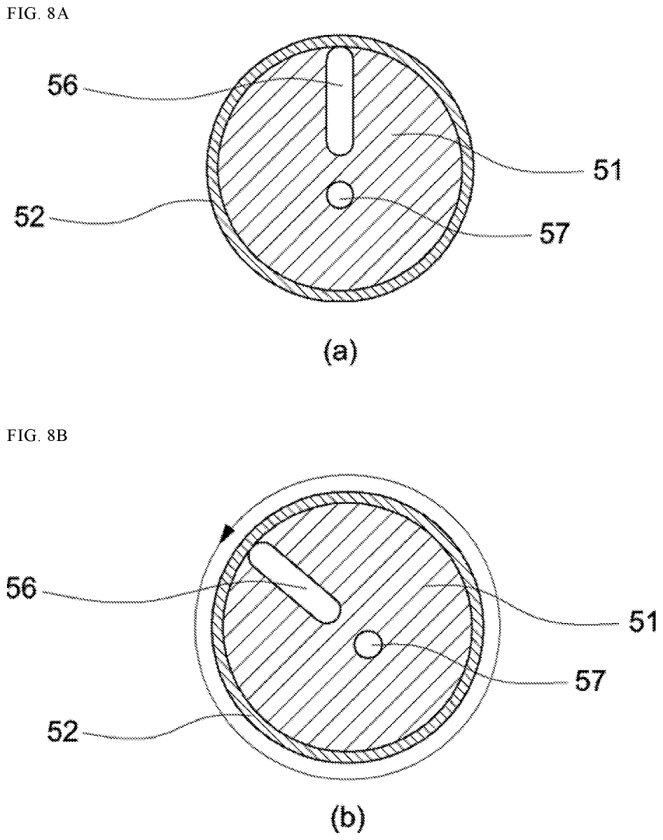

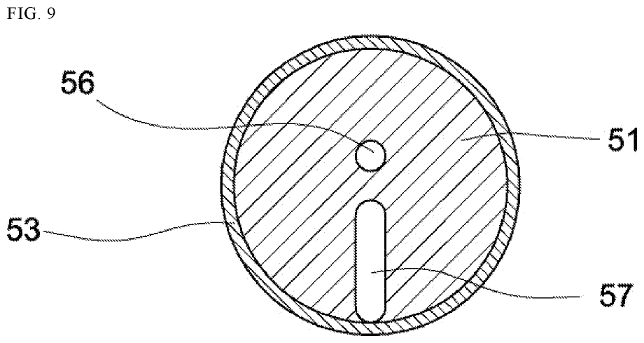

FIG. 1 is a view illustrating an overall appearance of a bipolar catheter according to the present disclosure, FIG. 2 is a view illustrating a decomposition configuration of an electrode unit, FIG. 3 is a view illustrating a front surface of the bipolar catheter illustrated in FIG. 1, FIG. 4 is a view schematically illustrating a cross-sectional configuration based on line A-A illustrated in FIG. 3, FIG. 5 is a view illustrating a state in which a guide portion slides forward based on FIG. 4, FIG. 6 partially illustrates a form in which an exposure length of an electrode tip is changed by moving an insulating cover forward by an operation of a roller member with respect to FIG. 4, FIG. 7 is an enlarged view of part B shown in FIG. 4, FIG. 8A is a view schematically illustrating a cross-sectional configuration based on line C-C illustrated in FIG. 7 and FIG. 8B is a view illustrating a state in which a cylinder illustrated in FIG. 8A is rotated at a certain angle, and FIG. 9 is a view schematically illustrating a cross-sectional configuration based on line D-D illustrated in FIG. 7.

As illustrated in FIGS. 1 to 4, the bipolar catheter according to the present disclosure may include a base portion 10, a guide portion 20 in which a driving motor 23 slidably installed in the base portion 10 and configured to generate rotational driving force is embedded, an electrode unit 30 which is mounted in an output terminal 24 of the driving motor 23 to rotate in conjunction with the driving motor 23 and is inserted into a lesion area to cauterize the lesion area, and a driver 40 which slides the guide portion 20 forward and backward in the base portion 10.

First, the base portion 10 forms a lowest portion of the bipolar catheter, and a guide rail 11 to which the guide portion 20 is connected may be formed on an upper surface of the base portion 10. In complement thereto, a guide groove 21a connected to the guide rail 11 may be formed on a lower surface of the guide portion 20.

The guide rail 11 and the guide groove 21a are only examples, and may be variously modified in the scope that can assist a smooth linear movement of the guide portion 20 on the upper surface of the base portion 10.

For example, a ball screw shaft, a linear shaft, and the like may be provided in another embodiment of the guide rail 11.

Next, the guide portion 20 may be guided to the guide rail 11 from the upper surface surface of the base portion 10 through the driver 40 while supporting the electrode unit 30 and may slide while reciprocating forward and backward.

That is, when the guide portion 20 slides forward and backward, the electrode unit 30 may also move in conjunction therewith by a distance at which the guide portion 20 has moved.

More specifically, the guide portion 20 may be formed integrally with a lower plate 21 connected to the upper surface of the base portion 10 and have the driving motor 23 installed therein, and may include a main body 22 configured to expose the output terminal 24 of the driving motor 23 in front, and a gripping clip 25 spaced apart from the front of the main body 22 and configured to grip an outer circumferential surface of the electrode unit 30.

The lower plate 21 forms a bottom portion of the guide portion 20, and the guide groove 21a may be recessed on a lower surface of the lower plate 21. In the upper portion of the lower plate 21, the main body 22 and the gripping clip 25 may be individually configured to be spaced apart from each other.

An electronic device such as the driving motor 23 and a high frequency generator (not illustrated) may be embedded in the main body 22. The driving motor 23 generates forward and reverse rotational driving forces according to a control signal and rotates the electrode unit 30 installed in conjunction with the driving motor 23.

A connector 50 may be mounted between the output terminal 24 exposed to the driving motor 23 and the electrode unit 30, and a current of the high frequency generator may be applied to the electrode unit 30 through the connector 50.

The structure of the connector 50 will be described below with a detailed description of the electrode unit 30.

The high frequency generator may be provided in any known structure capable of supplying a high frequency current toward the electrode unit 30.

The gripping clip 25 supports the electrode unit 30 directly connected to the driving motor 23 in the form of a cantilever beam. Specifically, when the electrode unit 30 rotated by the driving motor 23 is gripped from the outside, the rotation of the electrode unit 30 may be limited by the gripping clip 25, but this embodiment is configured to have a structure in which the entire electrode unit 30 does not rotate, but only an electrode body 31 which is an internal component of the electrode unit 30 rotates, thereby smoothly rotating an electrode tip 31a even in a state of being fitted into the gripping clip 25.

To this end, the electrode unit 30 may include an electrode body 31 to which a high frequency current is applied and in which one end thereof is provided with the electrode tip 31a heated by the high frequency current, an insulating cover 32 formed of a hollow body configured to accommodate the electrode body 31 therein and configured to expose the electrode tip 31a, and a fixing holder 33 formed of a hollow body configured to accommodate the other end of the insulating cover 32 therein and fitted to the gripping clip 25.

More specifically, one end of the electrode body 31 is disposed adjacent to the electrode tip 31a, and the other of the electrode body 31 may further include an active conductive wire 31b and a passive conductive wire 31c connected to the connector 50.

The active conductive wire 31b and the passive conductive wire 31c may be wound around, for example, an outer circumferential surface of the electrode tip 31a, and a plurality of active conductive wires 31b and a plurality of passive conductive wires 31c may be alternately disposed around the electrode tip 31a, if necessary.

In one embodiment of the present disclosure, one active conductive wire 31b and one passive conductive wire 31c are defined as being connected to the electrode tip 31a in a state in which the active conductive wire 31b and the passive conductive wire 31c are parallel to each other, thereby minimizing a whole outer diameter of the electrode tip 31a. Accordingly, the whole outer diameter of the electrode tip may be configured to be about 1.8 mm.

A current supplied from the high frequency generator is applied to the active conductive wire 31b and the passive conductive wire 31c, and is then radiated from the wound electrode tip 31a and used as thermal energy for cauterizing the lesion area. The active conductive wire and the passive conductive wire may be understood to have a structure corresponding to an electrode in a configuration of a known high frequency catheter.

That is, the high frequency generator has a structure of supplying the high frequency current to the electrode tip 31a through the active conductive wire 31b and the passive conductive wire 31c.

The electrode tip 31a may be formed in a needle shape to facilitate insertion into a lesion tissue. If necessary, the passive conductive wire 31c and the active conductive wire 31b may be accommodated in the insulating cover 32 in a state in which the passive conducting wire 31c and the active conducting wire 31b are covered with a separate outer shell, and in this case, the electrode tip 31a may be provided to get completely exposed from the outer shell.

The insulating cover 32 serves to protect the electrode body 31 accommodated inside, and to prevent high frequency from leaking to a portion of the electrode body 31 except for the electrode tip 31a of the electrode body 31.

On the other hand, assuming that cross sections of the electrode tip 31a and the insulating cover 32 is circular, an outer diameter of the electrode tip 31a may be formed smaller than an inner diameter of the insulating cover 32. Accordingly, the electrode tip 31a may be smoothly be inserted into or withdrawn from the insulating cover 32.

The insulating cover 32 is accommodated in the fixing holder 33, and the insulating cover 32 is configured to be moved forward and backward inside the fixing holder 33.

The other end of the electrode body 31 exposed to the other end of the insulating cover 32 may pass through an interior of the fixing holder 33 and may be connected to the connector 50.

The fixing holder 33 is a portion directly fitted into the gripping clip 25 and a portion of the electrode unit 30 except for the fixing holder 33 may be free from the gripping clip 25.

That is, the fixing holder 33 may be restricted to the gripping clip 25, and the electrode body 31 may freely rotate in a relative manner by the rotational driving force of the driving motor 23 inside the fixing holder 33. Accordingly, when the electrode body 31 rotates, since only the electrode tip 31a exposed to one end of the insulating cover 32, and the active conductive wire 31b and the passive conductive wire 31c in contact with the electrode tip 31a are rotated when viewed from the outside, the lesion tissue can be treated by accurately rotating the electrode tip 31a only at the target lesion area.

The insulating cover 32 may be usually inserted together into the body, and accordingly, if the electrode body 31 is rotated with the insulating cover 32, there may be a problem that the insulating cover 32 may apply external force to internal tissues of the body that do not require treatment.

In other words, in the embodiment of the present disclosure, since the electrode tip 31a is rotatable at the lesion area, even if the number of wires (e.g., active and passive wires) around the electrode tip 31a is reduced, high frequency may be effectively radiated to the lesion area. Accordingly, it is possible to insert the electrode tip 31a into a blood vessel with a small diameter by minimizing the diameter of the electrode tip 31a, and when inserting the electrode tip 31a from a central vessel into a branch vessel, or when inserting the electrode tip 31a into a heavily curved vessel, damage to a vascular wall can decrease by reducing a contact with the vascular wall.

Meanwhile, in the embodiment of the present disclosure, an exposure length of the electrode tip 31a may be adjusted to enable an elastic procedure without needing to replace electrode tips 31a having different sizes depending on the lesion area.

To this end, the electrode unit 30 may further include a roller member 34 that is rotatably mounted while penetrating through the outside of the fixing holder 33 and pulls the insulating cover 32 forward and backward as the roller member 34 as the roller member 34 rotates by coming into contact with an outer circumferential surface of the insulating cover 33 accommodated in the fixing holder 33.

As described above, the insulating cover 32 may be configured to move forward and backward in the fixing holder 33. In this state, when the roller member 34 is rotated forward and backward, the insulating cover 32 may be moved forward and backward by friction with the roller member 34 in the fixing holder 33.

A certain roughness may be formed on an outer circumferential surface of the roller member 34 to cause strong friction resistance against the outer circumferential surface of the insulating cover 32.

In other words, when the insulating cover 32 is moved forward and backward by simply rotating the roller member 34 without needing to replace the electrode tips 31a having different sizes, an exposure length of the electrode tip 31a may be changed by the insulating cover 32, and accordingly, depending on the area of the lesion, precise procedures can be achieved without needing to replace the electrode tip 31a.

Meanwhile, a stopper protrusion 32a caught by the roller member 34 may be formed in the other end of the insulating cover 32.

The stopper protrusion 32a is meant to restrict the insulating cover 32 from being completely removed to one end of the fixing holder 33 in the process of rotating the roller member 34 to move the insulating cover 32 forward and backward, and at a certain portion of the stopper protrusion 32a, the other end of the insulating cover 32 is hung on the roller member 34 and is no longer extended to one end of the fixing holder 33, thereby preventing the insulating cover 32 from being completely separated from the fixing holder 33.

On the other hand, in one embodiment of the present disclosure, when the electrode body 31 is rotated, a structure of a connector 50 is provided which prevents each of the conductive wires (e.g., the active conductive wire and passive conductive wire) from being twisted.

To this end, the connector 50 includes a cylindrical cylinder 51 provided with a second active terminal 54 mounted in the output terminal 24 of the driving motor 23 and connected to a first active terminal 31b′ formed on the other end of the active conducting wire 31b on the front surface and a second passive terminal 55 to which a first passive terminal 31c′ formed on the other end of the passive conducting wire 31c is connected, a conductive active band 52 and a passive band 53 wound around an outer circumferential surface of the cylinder 51, respectively, an active conductive pin 56 that is embedded in the cylinder 51 and extends from the second active terminal 54 to an inner circumferential surface of the active band 52 to form a contact point, and a passive conductive pin 57 that is embedded in the cylinder 51 and extends from the second passive terminal 55 to an inner circumferential surface of the passive band 53 to form a contact point.

More specifically, the cylinder 51 may be rotated by applying rotational driving force of the driving motor 23. Conversely, the active band 52 and the passive band 53 wound around the outer circumferential surface of the cylinder 51 may be fixed to a front surface of the main body 22 through a ‘¬’ shaped fixing bracket 54 without being rotated and interlocked with the cylinder 51

In other words, the cylinder 51 may be rotated in the center of the active band 52 and the passive band 53. Furthermore, when the cylinder 51 is rotated, the electrode body 31 itself may be rotated therewith.

The active band 52 and the passive band 53 may be electrically connected to a high frequency generator and receive a current.

Each of the active band 52 and the passive band 53 may be formed of a cylindrical band having a metal material.

The active conductive pin 56 and the passive conductive pin 57 may form contact points with the active band 52 and the passive band 53, respectively, and receive a high frequency current.

As illustrated in FIGS. 7 to 9, in the active conductive pin 56, one end thereof may be connected to the second active terminal 54, and the other end thereof may be in contact with the inner circumferential surface of the active band 52 by bending vertically a path toward the active band 52.

In the same way as above, in the passive conductive pin 57, one end thereof may be connected to the second passive terminal 55, and the other end thereof may be in contact with the inner circumferential surface of the passive band 53 by bending vertically a path toward the passive band 53.

So configured, when the connector 50 is rotated by the driving motor 23 in a state in which the other end of the active wire 31b and the passive wire 31c is restrained by the connector 50, the cylinder 51 is relatively rotated inside the active band 52 and the passive band 53, and at the same time, the active conductive pin 56 and the passive conductive pin 57 may be rotated in a circular orbit while maintaining contact points with the active band 52 and the passive band 53, respectively, thereby continuously supplying power to the electrode body 31, and preventing the active conductive wire 31b and the passive conductive wire 31c from twisting each other.

That is, when the electrode body 31 can have a detachable configuration on the connector 50 and is specifically mounted in the connector 50, the electrode body 31 may receive rotational force of the connector 50 and a current supplied from the connector 50.

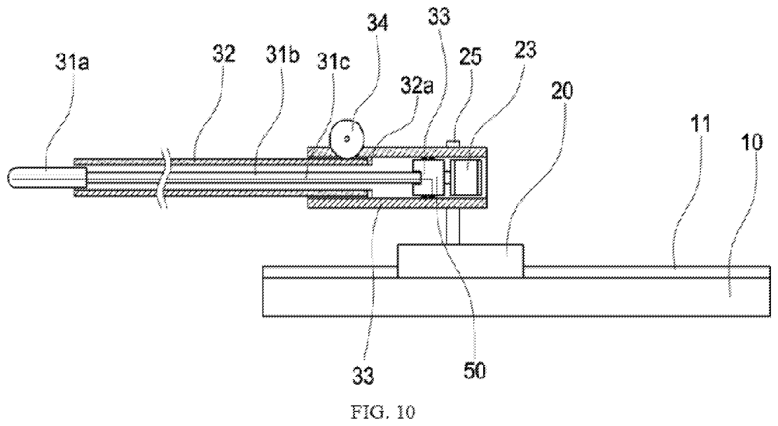

Hereinafter, another embodiment of a bipolar catheter according to the present disclosure will be described with reference to FIG. 10.

The bipolar catheter in one embodiment of the present disclosure has the same configuration as the bipolar catheter in the above-described embodiment, but is partially different in that the driving motor 23 is accommodated in the fixing holder 33.

That is, in the above-described embodiment, the driving motor 23 is embedded in the main body 22, but in this embodiment, the main body 22 is omitted and the connector 50 and the driving motor 23 are installed inside the fixing holder 33. Accordingly, the guide portion 20 has a structure in which the configuration of the main body 22 is omitted.

Hereinafter, an operation of the present disclosure will be described. In the operation, a process of treating vascular disease using the bipolar catheter is briefly described as an example.

First, an an electrode hole is formed using an 18-gauge vasoconstrictor needle in a vein of a patient lying on a bed, and the electrode unit 30 is inserted slowly from the electrode tip 31a. In this case, when the electrode unit 30 is inserted to a target part of the lesion area, the electrode tip 31a and the insulating cover 32 may be substantially inserted into the lesion area, and for convenience, this is referred to a process of inserting the electrode unit 30.

The process of inserting the electrode unit 30 may be performed by a remote operation of the driver 40. For example, when the driver 40 is controlled to slide the guide portion 20 forward from the base portion 10, the electrode unit 30 may be slowly inserted into the lesion area by interlocking therewith.

An operator precisely controls the driver 40 to remotely control an insertion depth of the electrode unit 30 into the lesion area. In this case, a control means for controlling the driver 40 may be provided.

In other words, the insertion depth of the electrode tip 31a into the lesion area may be controlled according to a distance in which the guide portion 20 slides forward and backward.

In this way, by electronically controlling the sliding amount of the guide portion 20, safer and more accurate procedures can be achieved as compared to the conventional procedure method that relied on the operator's medical treatment.

Meanwhile, the driver 40 may be understood as a means such as a known linear motor capable of precisely controlling the amount of movement of a specific member.

When the electrode tip 31a reaches the target part of the lesion area, the high frequency generator applies high frequency to the electrode tip 31a. At the same time, when the driving motor 23 rotates the electrode body 31, the high frequency is evenly radiated around the electrode tip 31a and treats the lesion area.

In summary, the bipolar catheter according to the present disclosure restrains the fixing holder in the gripping clip and allows the electrode body to freely rotate in the relative manner by the rotational driving force of the driving motor inside the fixing holder, whereby if the electrode body rotates, only the electrode tip exposed to one end of the insulating cover may rotate when viewed from the outside, thereby treating the lesion by accurately rotating the electrode tip only at the target lesion area. Therefore, the more precise procedure may be achieved, and damage to surrounding tissues that may occur during the invasive procedure using the catheter may be minimized.

In addition, by electronically precisely controlling the position of the electrode tip in the lesion area, safer and more accurate procedures can be ensured as compared to the conventional procedure method that relied on the operator's medical treatment.

Furthermore, electrode tips have different sizes do not need to be replaced, and when the roller material rotates to move the insulating cover forward and backward, since the exposure length of the electrode tip varies by the insulating cover, precise procedures can be achieved depending on the area of the lesion without needing to replace the electrode tip. Accordingly, the convenience of procedure may be greatly improved, and peripheral devices of the catheter may be greatly simplified.

When the connector is rotated by the drive motor in a state in which the other end of the active wire and the passive wire is restrained by the connector, the cylinder is relatively rotated inside the active band and passive band, and at the same time, since the active and passive wires can be rotated in the circular orbit while maintaining contact points with the active band and the passive band, power supply can be continuously performed and the active wire and the passive wire can be prevented from twisting each other.

Although embodiments of the present disclosure have been described for illustrative purposes, those skilled in the art will appreciate that various changes and other equivalent embodiments can be made. Therefore, it will be understood that the present disclosure is not limited only to the form mentioned in the detailed description. Accordingly, the true technical protection scope of the present disclosure will be defined by the technical spirit of the appended claims. In addition, it should be appreciated that the present disclosure is intended to include all modifications and equivalents and alternatives falling within the spirit and scope of the disclosure as defined by the appended claims.

1. A bipolar catheter for a vascular treatment, comprising:
a base portion;
a guide portion installed slidably in the base portion and equipped with a driving motor configured to generate rotational driving force;
an electrode unit configured to rotate in conjunction with the driving motor and inserted into a lesion area to cauterize the lesion area; and
a driver configured to slide the guide portion in front and rear directions in the base portion.

2. The bipolar catheter for a vascular treatment of claim 1, wherein the guide portion comprises:
a main body integrated with a lower plate connected to an upper surface of the base portion and equipped with the driving motor, wherein an output end of the driving motor is exposed to the front; and
a gripping clip spaced apart from the front of the main body and configured to grip an outer circumferential surface of the electrode unit.

3. The bipolar catheter for a vascular treatment of claim 2, wherein the electrode unit comprises:
an electrode body to which a high frequency current is applied and in which an electrode tip heated by the high frequency current is formed in one end thereof;
an insulating cover formed of a hollow body accommodating the electrode body inside and configured to expose the electrode tip; and
a fixing holder formed of a hollow body accommodating the other portion of the insulating cover inside and fitted and inserted into the gripping clip.

4. The bipolar catheter for a vascular treatment of claim 3, wherein the depth of an insertion of the electrode tip into the lesion area is controlled depending on a sliding distance of the guide portion in front and rear directions.

5. The bipolar catheter for a vascular treatment of claim 3, wherein an outer diameter of the electrode tip is formed smaller than an inner diameter of the insulating cover.
